(19) United States
(12) Patent Application Publication
(10) Pub. No.: US 2012/0179176 A1
(43) Pub. Date: Jul. 12, 2012

(54) APPARATUS AND METHOD FOR LIMITING SURGICAL ADHESIONS

(75) Inventors: Jeffrey A. Wilson, Wrentham, MA (US); Michael T. Milbocker, Holliston, MA (US)

(73) Assignee: PROMETHEAN SURGICAL DEVICES, LLC, East Hartford, CT (US)

(21) Appl. No.: 13/337,393

(22) Filed: Dec. 27, 2011

Related U.S. Application Data
(60) Provisional application No. 61/427,596, filed on Dec. 28, 2010.

(51) Int. Cl. A61B 17/03

(52) U.S. Cl. 606/151

(57) ABSTRACT

The present invention relates to a composite prosthesis including a coated mesh having at least one opening through a first surface and a second surface of the coated mesh; the coated mesh comprising a mesh and a biocompatible coating substantially surrounding each filament of the mesh, wherein the biocompatible coating is formed by coating the mesh with a polyol prepolymer and curing the prepolymer, the prepolymer comprising a polyalkylene oxide polyol end capped with isocyanate, the polyalkylene oxide polyol having from about 70% to about 95% ethylene oxide groups and the remainder propylene oxide; and a barrier material comprising a biocompatible membrane constructed and arranged to cover at least one surface of the coated mesh, wherein the barrier material comprises a biologic material.

### RELATED APPLICATIONS

The present application claims the benefit of priority to U.S. Provisional Patent Application No. 61/427,596, filed Dec. 28, 2011, the disclosure of which is hereby incorporated by reference.

### FIELD OF THE INVENTION

Disclosed herein are implantable composite prosthesis and method for limiting the incidence of acute postoperative adhesions and calcified scar formation embedded in the prosthesis that can result in a lifetime of post surgical complications and in particular post-operative complications in the field of visceral or parietal soft tissue repair surgery.

### BACKGROUND OF THE INVENTION

Post surgical adhesions include all non-anatomical fibrous connections accidentally induced by a surgical act during the normal process of cicatrization and may occur in all surgical disciplines regardless of the operation in question. Adhesions can provoke syndromes which can be classed principally as but not necessarily limited to chronic pain, occlusive syndromes, intestinal obstructions and female infertility. Therefore, it is evident that there is a need for a suitable method for preventing the adhesions, and the complications and patient discomfort associated with them.

One solution to reduce acute adhesion consists of separating adjacent internal bodily tissues by interposing a reinforcement or surgical mesh and barrier layer prosthesis so that during tissue regeneration following surgery no contact exists between the repaired tissue and surrounding organs or other tissue. However, the desired barrier effect of a non-absorbable barrier material can itself be the source of adhesions over the course of time as experienced with current composite prosthesis comprising a barrier made of expanded PTFE (Composix® EX Mesh, Davol/BARD®, Cranston, R.I.); and if it is an absorbable barrier, its absorption must be sufficiently non-inflammatory so as not to be a cicatrizant and cause adhesions itself. In the field of internal medical care, such as internal surgery, there is a need for tissue regeneration devices which may prevent complications such as adhesions in the post-operative healing period.

The approach of utilizing a barrier material is used in U.S. Pat. No. 5,002,551 which discloses a physical barrier formed of a knitted oxidized regenerated cellulose. The patent identifies other physical barriers including silicone elastomers and absorbable gelatin films. Such physical barriers alone are not sufficient to reinforce the abdominal wall or to repair abdominal wall defects.

As recognized in the art, e.g., for visceral and parietal surgery, but also in orthopedic or neurological surgery, the composite prosthesis must also have a certain mechanical strength and permanence allowing it to fulfill a lifetime function as an element of surgical reconstruction. Generally, the known prosthetic fabrics or meshes, e.g., in the treatment of parietal insufficiencies, for example hernias, other eventrations and organ suspensions, afford additional mechanical strength to the surgical reconstruction. Most surgical mesh used in the art have the characteristic of being densely knitted and constructed of a monofilament fiber or multifilament yarn so as to present a significant number of very small interstices caused by the knots or loops of the mesh that increase surface area and promote tissue in-growth of scar tissue inside these interstices. Even though scar formation has a purpose in tissue healing, clinically calcified scar tissue is considered pathological and suboptimal to connective tissue that can support blood vessels and act like normal native tissue. Unfortunately, current surgical mesh is constructed of synthetic materials that react with normal tissue invoking a chronic inflammation response and calcified scar encapsulating the mesh prosthesis. It is for this reason that upon contact with the viscera for example, these fabrics promote adhesion which is a feature that limits their use at the so-called preperitoneal or retroperitoneal sites. With some patients the mesh has to be removed due to the complications of scar pathology. In certain procedures, including incisional and umbilical hernia repair and chest reconstruction, the prosthetic mesh may come into direct contact with the sensitive abdominal viscera, creating postoperative adhesions between the mesh and the intestine, potentially leading to intestinal fistulization

Because of the shortcomings of a non-absorbable mesh only approach, various approaches to reducing the incidence of postoperative adhesions during healing or chronic scaring arising from the use of prosthetic mesh materials have been used. One traditional solution is to cover the prosthesis with peritoneum or other tissue, where available or adequate to close the defect, to form a biological barrier between the implant and the bowel. Another solution includes the placement of a physical barrier between the surgical site and the surrounding tissue where adhesions are most commonly encountered.

Absorbable combinations of a mesh and other materials or barriers, such as those disclosed in U.S. Pat. No. 4,840,626 and U.S. Pat. Pub. No. 2005/0283256, have been disclosed for use, however there are surgical situations that benefit from having a mesh prosthesis that is not totally absorbed and therefore adds strength to a wound area after it heals.

Barriers having multiple layers, one of which is porous, are disclosed in U.S. Pat. Nos. 5,508,036 and 5,480,436. These barriers are helpful, however there are additional benefits provided by the strength provided by a mesh fabric.

One approach to address the above problems is to combine the features of a non-absorbable mesh with an absorbable barrier sheet. Jenkins et al., “A Comparison of Prosthetic Materials Used to Repair Abdominal Wall Defects”, Surgery, Vol. 94, No. 2, August 1983, pg. 392-398, describes a technique of placing an absorbable gelatin film (GELFILM®) between a piece of Marlex knitted polypropylene monofilament mesh and the abdominal viscera. U.S. Pat. No. 6,451,032 describes a multi-layer prosthesis with one embodiment comprising a mesh and a collagenous material. U.S. Pat. No. 5,593,441 discloses a prosthesis comprising a mesh and an absorbable barrier. However, with these solutions, either through initial contact with the wound, or contact once the barrier is absorbed into the body, the uncoated mesh material stimulates in-growth of scar tissue into and around the mesh. In this case, in-growth is defined as a growth of tissue to or into a fabric, mesh or similar device, connecting an artificial surface to living tissue, but not necessarily extending through it. This in-growth results in fibrotic tissue that, through the fibrotic healing process, is eventually reabsorbed by the body. As a result of this reabsorption, the fibrotic layer formed on the mesh contracts in the direction normal to the mesh and in the plane of the mesh. As the layers contract they pull the mesh with it, causing it to fold and buckle. The result is usually a hard and painful locus of tissue and implant which also increases the instance of adhesion with surrounding tissues.

Microscopic examination of tissue in-growth in both polyester and polypropylene mesh suggests it is the inflammatory potential of the mesh that promotes fibrosis along the plane of the mesh. Therefore, to reduce fibrosis, it is beneficial to reduce the in-growth and inflammatory potential of the mesh.

As opposed to in-growth in the knots or loops of the mesh, tissue through growth is a healing method that can only be promoted by preventing scar tissue forming in the interstices and promoting connective tissue growth through much larger windows or openings in the mesh fabric. This continuous tissue connection extends through the fabric or mesh or other artificial surface from one living tissue to another. In the case of a mesh and barrier combination, through-growth is confined to tissue connections between adjacent points on the layer of tissue surgically attached to the mesh. Connective tissue through-growth, promoted by the plurality of the larger window pane design, or openings of the mesh, helps decrease the likelihood of infections by preventing microbe proliferation along the surface of densely woven or knitted mesh. Through growth also does not have the detrimental effects of fibrotic healing related to in-growth. Through-growth supports angiogenesis and further fights infections.

Hydrogels are a material that has a reduced potential for inflammation, and therefore fibrosis, in a tissue. Hydrogels are uniquely biocompatible and contain large amounts of loosely bound water that is free to equilibrate in osmolarity and chemical composition with the surrounding tissue. This exchange of the hydrogel water with the surrounding tissue water makes prosthetics made from hydrogel more tissue-like and hydrophilic, and discourages the attachment of protein markers on the surface of the prosthetic. These features dramatically reduce the inflammatory potential of the prosthetic and reduce the promotion of fibrosis. However, hydrogel-based prosthetics are not currently used in surgical soft tissue repair, primarily because such prosthetics are expected to provide permanent tissue support and most hydrogels are either absorbable or possess little tensile strength.

U.S. Pat. No. 5,593,441 discloses a method for limiting the incidence of postoperative adhesions. A composite of a mesh and a barrier is positioned with the barrier facing away from the defect wall opening. The mesh has a plurality of interstices constructed and arranged to allow tissue in-growth.

U.S. Pat. Pub. No. 2006/0233852 discloses hydrogels reinforced with mesh for use in the repair of tissue defects such as a hernia in order to reduce the incidence of adhesions. The entire contents of U.S. Pat. Pub. No. 2006/0233852 is incorporated herein by reference as if repeated in full herein.

### SUMMARY OF THE INVENTION

Disclosed herein are composite prostheses and methods for reinforcing and repairing a weakened tissue defect while limiting the incidence of postoperative adhesions and calcified scar formation. in one embodiment, the composite prosthesis is formed of a biologically compatible or biocompatible, flexible and porous implantable mesh suitable for reinforcing tissue and closing tissue defects, e.g., in the abdominal cavity, a biologically compatible coating to cover the mesh and a barrier material for physically isolating the tissue defect site from the implantable mesh and areas likely to form adhesions, such as the abdominal viscera. The coated mesh and barrier material are combined by means of incorporating both elements into a single layer fabric or by attaching both materials through means of an adhesive, coating, stitching or insert molding. The barrier material is absorbable or otherwise degradable in a manner which allows tissue through-growth to anchor the implantable mesh material through openings in the implantable mesh.

In one embodiment of the invention, the implantable material comprises at least one sheet of knitted polypropylene monofilament mesh fabric, or similar materials, coated with a hydrogel.

In another embodiment of the invention, the coating closes the knotted or looped interstices and completely surrounds the multifilament structures of the mesh to ensure that scar tissue does not form in the small interstices and the coating also reduces the total mesh surface area exposed to tissue compared to an uncoated surface. In one embodiment, the biocompatible character of the hydrogel coating reduces the negative tissue reactions to polypropylene, polyester or other synthetic materials of uncoated meshes.

One embodiment provides a composite prosthesis comprising a coated mesh having at least one opening through a first surface and a second surface of the coated mesh, the coated mesh comprising a mesh and a biocompatible coating substantially covering the mesh. In one embodiment, the barrier material comprises a biologic material. In one embodiment, the mesh comprises a coated knitted mesh and the barrier material is attached to at least one surface of the coated mesh where the coating is formed by curing a polyalkylene oxide polyol end capped with isocyanate, the polyalkylene oxide polyol having from about 70% to about 95% ethylene oxide groups and the remainder propylene oxide.

Another embodiment provides a composite prosthesis comprising a coated mesh having at least one opening through a first surface and a second surface of the coated mesh, the coated mesh comprising a mesh and a biocompatible coating substantially covering the mesh and a barrier material comprising a biocompatible membrane constructed and arranged to cover at least one surface of the coated mesh. In one embodiment, the barrier material comprises a poly lactide polymer or co-polymer In one embodiment, the coated mesh comprises a knitted mesh and the barrier material is attached to at least one surface of the coated mesh, i.e., one or both surfaces of the coated mesh.

Another embodiment provides a composite prosthesis which combines the attributes of a surgical mesh fabric and of a physical barrier.

Another embodiment provides a composite prosthesis for repairing ventral hernias and for reconstructing the chest wall which limits the incidence of postoperative adhesions and intestinal fistulization.

Another embodiment provides a composite prosthesis which minimizes inflammatory stimuli to the tissue surrounding the surgical opening and minimizes the inflammatory response of other areas of potential adhesions such as the abdominal viscera.

Another embodiment provides a composite prosthesis which provides an implantable material that is retained near the surface of the tissue opening to continue to reinforce the tissue wall.

Another embodiment provides a composite prosthesis which may be custom shaped, sized and affixed during surgery without destroying the integrity of the device.

Another embodiment provides a prosthesis which is sufficiently flexible to conform to the surgical site.

Another embodiment provides methods of utilizing embodiments of a composite prosthesis that limits the incidence of postoperative adhesions.

Another embodiment provides a method for limiting the incidence of postoperative adhesions arising from a repair of a defect in a tissue comprising the steps of providing a composite prosthesis comprising a coated mesh and a barrier and positioning the composite prosthesis to cover the defect whereby the formation of postoperative adhesions is limited.

Another embodiment provides a method of limiting the incidence of postoperative adhesions arising from a repair of a defect in a tissue comprising the steps of providing a composite prosthesis comprising a coated mesh and a barrier and positioning the composite prosthesis with the coated mesh away from the defect and the barrier material positioned between defect and the coated mesh whereby the formation of postoperative adhesions is limited.

Another embodiment provides a method of making a composite prosthesis comprising the steps of coating at least one fiber with a biocompatible coating to form a coated mesh and attaching an adhesion-resistant barrier material to the coated mesh.

Another embodiment provides a composite prosthesis in which the barrier material comprises a biologic material.

Other embodiments of the present invention will become apparent from the following detailed description.

### BRIEF DESCRIPTION OF THE DRAWINGS

Various embodiments of the invention will be understood from the following description, the appended claims and the accompanying drawings, in which:

FIG. 1A is a top perspective view of one embodiment of the composite prosthesis.

FIG. 1B is a top view of one embodiment of a mesh.

FIG. 1C is a top view of one embodiment of a coated mesh.

FIG. 2A is an isometric view of one embodiment of the composite prosthesis with the mesh and barrier material attached with a portion of the barrier material held away from the mesh.

FIG. 2B is an isometric view of one embodiment of the composite prosthesis with the mesh and the barrier material not attached.

FIG. 3A is a cross-section view of one embodiment of the composite prosthesis showing the mesh and barrier material attached by a biocompatible coating.

FIG. 3B is a top view, in partial section view, of one embodiment of the composite prosthesis with a portion of the barrier material removed showing the mesh and barrier material attached by an adhesive.

FIG. 3C is a top view, in partial section view, of one embodiment of the composite prosthesis with a portion of the barrier material removed showing the mesh and barrier material attached by an adhesive.

FIG. 4 is a side, perspective view of one embodiment of the composite prosthesis and its positioning relative to a tissue defect.

FIG. 5 is a side cut-away view of one embodiment of the composite prosthesis illustrating one proposed position of the composite prosthesis positioning relative to a tissue defect and surrounding organs.

FIG. 6 is a process diagram illustrating one embodiment of a method of using the composite prosthesis.

FIG. 7 is a process diagram illustrating one embodiment of a method of making the composite prosthesis.

FIG. 8 is an isometric view, with parts separated, of a coated mesh partially covered by barrier material.

FIG. 9 is a top view of a coated mesh partially covered by a barrier material.

### DETAILED DESCRIPTION

Disclosed herein are composite prostheses that can be useful in parietal surgery, in the repair of eventrations or hernias. These descriptions are used as examples of embodiments and use of these embodiments of the invention and are not intended to limit embodiments or uses.

### The Apparatus:

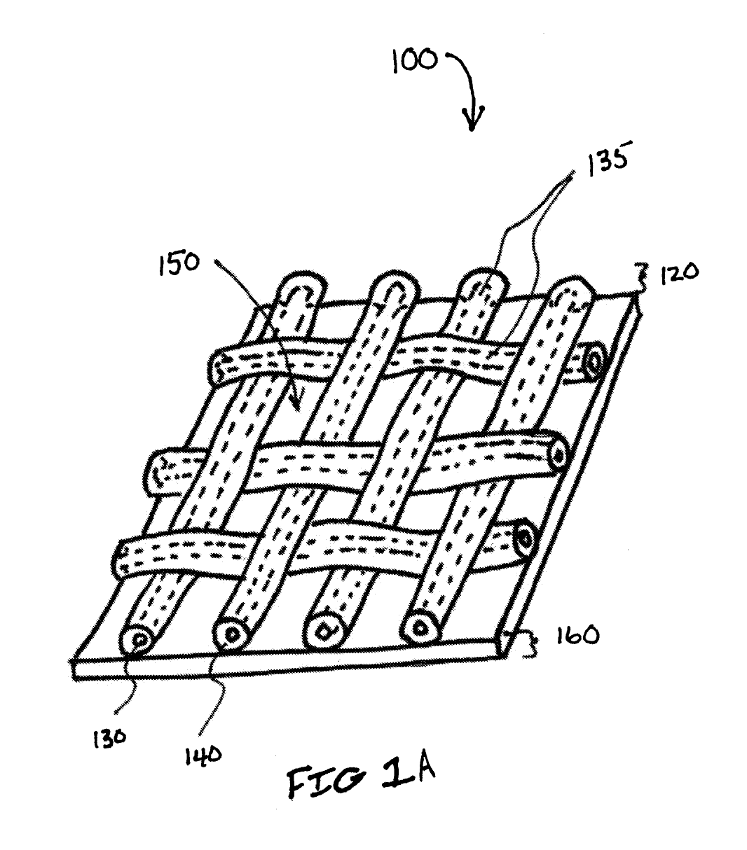

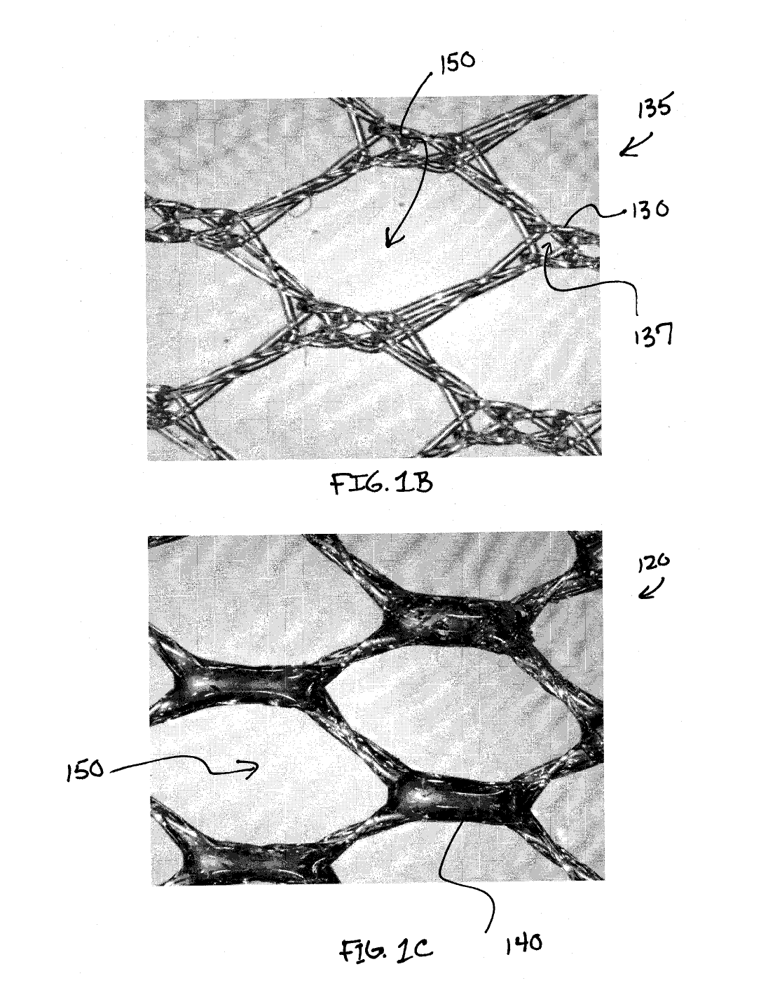

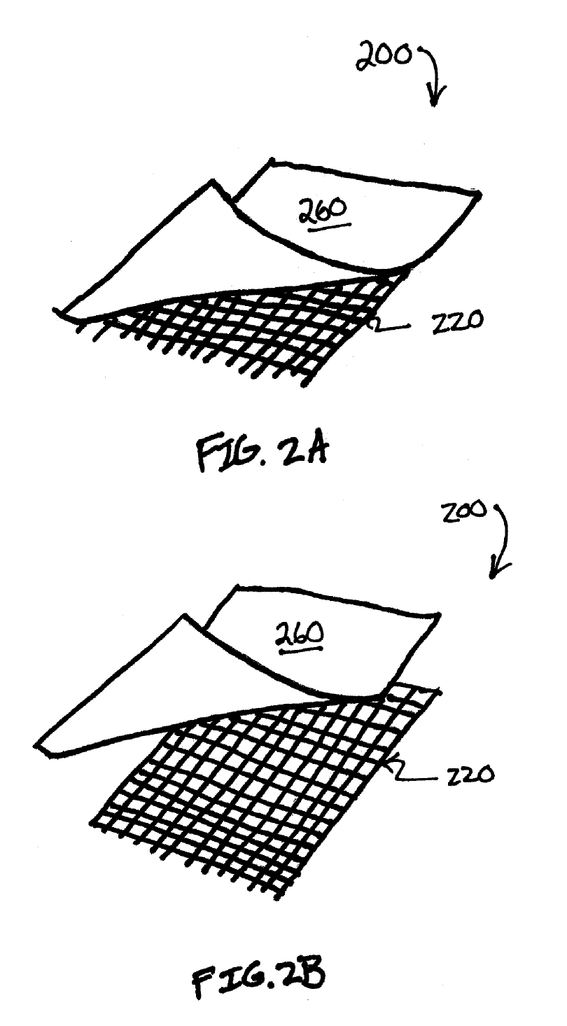

Referring to FIG. 1A, the composite prosthesis 100 for limiting the incidence of postoperative adhesions includes a tissue infiltratable mesh 135 comprising one or more fibers 130 coated with a biocompatible coating 140 creating a coated mesh 120. The coated mesh 120 is fully or partially covered on one or both sides by an adhesion resistant bioabsorbable barrier material 160. The coated mesh 120 construction creates a plurality of pores, windows or openings 150 which are of sufficient size and orientation to allow sufficient tissue through-growth to secure the composite prosthesis 100 to a defect site once the stimulus for tissue adhesion formation has subsided and the barrier material 160 has been absorbed. The integration of the barrier material 160 and the coated mesh 120 separates the tissue defect from the area of potential tissue adhesion. The composite combines the strength, handling and adhesive properties of a prosthetic mesh with the low adhesion incidence of a physical barrier.

The term “biocompatible” as used herein refers to biologically compatible materials that do not elicit a toxic or severe immunological response following implantation or ingestion in a body or other organism.

The term “bioabsorbable” as used herein encompasses the complete resorption of the materials of the apparatus by the body as well as a breakdown of the structure of the apparatus without complete resorption of the apparatus; i.e., the structure of the apparatus is broken down into a plurality of pieces which are not completely resorbed.

The relatively flat composite prosthesis 100 is sufficiently pliable to allow a surgeon to manipulate the shape of the prosthesis to conform to the anatomical site of interest and to be sutured, glued, tacked or stapled there. Alternatively, the composite prosthesis 100 may be pre-formed into a more complex shape, such as a tapered plug for filling and occluding a ruptured wall. The shape and size of the composite prosthesis 100, and of the respective coated mesh 120 and barrier material 160, may vary according to the surgical application as would be apparent to those of skill in the art.

### The Apparatus Mesh and Mesh Coating:

Referring to FIG. 1A, in one embodiment, the mesh 135 is formed from a knitted fabric that contains openings 150. A suitable fabric for the mesh 135 includes a sheet of knitted polypropylene monofilament mesh fabric such as MARLEX® mesh available from C. R. Bard, Inc. Other surgical materials suitable for the mesh 135 include, but are not limited to PROLENE®, DACRON®, TEFLON®, MERSELEN® and PARIETEX™ by Covidien. It also is contemplated that the mesh 135 may be formed from porous membranes, multifilament yarns and that woven, molded and other recognized methods of forming a prosthetic mesh with openings, windows or pores are suitable. It is also contemplated that the mesh 135 may be formed from materials such as fibers and non-porous membranes that when coated with a biocompatible coating, as described below, are capable of possessing the features of porous membranes or prosthetic meshes. Examples of suitable materials for the mesh 135 include, but are not limited to those described in at least U.S. Pat. Nos. 3,054,406; 3,124,136; 4,193,137; 4,347,847; 4,452,245; 4,520,821; 4,633,873; 4,652,264; 4,655,221; 4,838,884; 5,002,551; and European Patent Application No. 334,046 all of which are incorporated by reference. Monofilament and multifilament polyester mesh materials are also contemplated.

FIG. 1B illustrates one embodiment of a mesh 135 showing the openings 150 of the mesh, the mesh fibers 130 and interstices 137 formed in the mesh 135.

Referring to FIG. 1A, the fibers 130 of the mesh 135 can be coated with a non-absorbable, biocompatible coating 140 such as a hydrogel creating a coated mesh 120. This coating 140 reduces the inflammatory reaction caused by the coated mesh 120 against tissue. When mixed with an aqueous solution, the hydrogel encapsulates the fibers 130 of the mesh 135. The mesh 120 with a coating 140 can be made such that no portion of the fibers 130 are exposed beyond the coating 140. Thus the chemical and physical composition of the fiber 130 does not necessarily contribute to a tissue response when placed in a mammalian body.

FIG. 10 illustrates one embodiment of a coated mesh 120 showing the openings 150 of the coated mesh 120 and the biocompatible coating 140 covering the mesh fibers and the interstices of the mesh.

In one embodiment, the coating 140 is a hydrogel. In one embodiment, a hydrogel for coating the mesh 135 is the surgical adhesive described in U.S. Pat. Pub. No. 2005/0129733, which is herein incorporated by reference in its entirety.

Examples of other suitable coatings 140 include, but are not limited to those non-absorbable prepolymers described in at least U.S. Pat. No. 4,990,357, U.S. Pat. Pub. No. 2002/0049503 and U.S. Published. Application No. 2005/0129733 all of which are incorporated by reference in their entirety. A hydrogel invites little fibrosis, because there is little protein absorption which is involved in cell attachment and, hence, little fibrosis. The hydrophilicity of the coating prevents fibrous tissue adhesion directly to the surface of the coating and promotes un-stimulated tissue growth around this coating. Unstimulated tissue growth allows the through-growth of tissue into the openings of the coated mesh 120 which is different than normal in-growth into the interstices of the mesh. Through-growth does not adhere or scar the tissue to a foreign surface such as the mesh and therefore does not promote fibrosis along the plane of the mesh. Through growth is a continuous tissue to tissue connection extending through or around a foreign surface without attaching to the foreign surface.

In one embodiment of the composite prosthesis 100, the coating 140 comprises a non-absorbable hydrogel. Suitable non-absorbable hydrogel compositions suitable for the mesh 135 coating are described in U.S. Pat. No. 6,296,607, in U.S. Published. Application No. 2003-0135238, and in U.S. Published. Application. No. 2005-0215748 each of which is incorporated by reference in their entirety. Other types of hydrogels that may be used in embodiments of the invention. Some of these other types are described in U.S. Pat. No. 5,410,016 which is herein incorporated by reference.

Prepolymers of polyurethanes form one embodiment of hydrogels used as coating 140 in one embodiment of this invention. These prepolymers are formed by endcapping triols, or triolized diols, with low molecular weight diisocyanate, and then reacted the product of these steps with an excess of water. When the polyol component is a polyalkylene oxide (PAO) constructed from approximately 75% (70%-95%) ethylene oxide monomers and about 25% (5%-30%) propylene oxide monomers, the resulting hydrogel can contain up to 90% water and achieve desirable stability and strength characteristics. The PAO can be made as a diol (two armed) and later made capable of crosslinking by trimerization with a low molecular weight triol (such as trimethylol propane, TMP) or a higher-functionality material. The PAO can also be made as a triarmed structure by starting with a trifunctional starter, such as TMP.

One embodiment of prepolymers are the product of reacting about 20% by weight to about 40% by weight TDI (toluene diisocyanate), 65% by weight to about 85% by weight polyalkylene oxide (PAO) diol and about 0.5% by weight to about 2% by weight TMP (trimethylol propane). In one embodiment, the composition is the product of reacting in weight ratios about 20% to about 25% TDI, 70% to about 80% PAO diol and about 0.7% to about 1.2% TMP. In other embodiments, the composition is the result of reacting about 23% to about 25% TDI, about 73% to about 77% diol and about 0.7% to about 1.0% TMP. In other embodiments, the composition is the result of reacting about 24% TDI, 75% diol and about 0.7% to 1.0% TMP. In the above reaction products, the diol can have values in the range of about 70%-95% ethylene oxide monomers and 5%-30% propylene oxide monomers, e.g., 75% polyethylene glycol and 25% polypropylene glycol.

Other embodiments are the product of reacting about 20% by weight to about 40% by weight IPDI (isophorone di-isocyanate; an aliphatic diisocyanate with a slower reaction rate than TDI), 65% by weight to about 85% by weight diol and about 1% by weight to about 10% by weight TMP. In certain embodiments, the composition is the product of reacting in weight ratios about 25% to about 35% IPDI, 70% to about 80% diol and about 2% to about 8% TMP. In other embodiments, the composition is the result of reacting about 25% to about 30% IPDI, about 70% to about 75% diol and about 1% to about 8% TMP. In yet other embodiments, the composition is the result of reacting about 25% IPDI, 70% diol and about 1% to 2% TMP. In the above reaction products, an exemplary diol is 75% polyethylene glycol and 25% polypropylene glycol.

In one embodiment of a hydrogel-forming composition described above, the coating material is a polyol prepolymer containing a polyalkylene oxide polyol end capped with isocyanate. In one embodiment, the polyol is tri-functional as described, and polyalkyene oxide monomers in the composition consist of from about 70% to about 95% ethylene oxide monomer with the rest of the monomers being propylene oxide.

Other non-absorbable, biocompatible materials may be utilized as the coating 140 as would be apparent to those of skill in the art, the ultimate selection depending upon the composition of the mesh 135 and the barrier material 160.

It is contemplated that the embodiments of the coated mesh include coating the fibers or other material of the mesh prior to creating the mesh as well as coating the fibers or other materials of the mesh after they are formed as the mesh. For example, single fibers can be coated with the biocompatible coating to form a coated mesh as well as using single fibers to create a mesh that can then be coated with a biocompatible coating to form a mesh.

### The Apparatus Barrier Material:

Referring to FIG. 1A, the barrier material 160 comprises an anti-adhesive bioabsorbable segmented copolymer comprised of a bioabsorbable, biodegradable or bioerodable polymer. In one embodiment, the barrier material comprises a biologic material, as described herein. In one embodiment, the barrier material 160 is formed as a generally planar, non-porous (i.e., essentially having no pores) membrane which acts as a barrier layer. In one embodiment, the barrier material 160 is non-porous however it is also contemplated that pores can be present in the barrier material 160 provided that the pores do not interfere with the barrier material's ability to reduce the incidence of adhesion. When pores are present, in one embodiment the pores of the barrier material 160 are less than 3 microns. The barrier material 160, in general, also prevents the in-growth of tissue and has a thickness of from about 5 microns to about 300 microns, e.g., from about 10 microns to about 300 microns.

The barrier material 160 is bioabsorbable, biodegradable, or bioerodable, i.e., the barrier material is broken down gradually by the body after implantation. After a period of time, which may vary depending upon various factors such as the thickness of the barrier material 160 layer, the proportion of the components of the polymer, and the specific use of the polymer, the polymer loses its unitary structure. For example, the polymeric device breaks into pieces, and may eventually be completely resorbed. In one embodiment, the polymer is bioabsorbable in addition to being biodegradable; i.e., the polymer is resorbed by the body such that the polymeric device becomes essentially non-detectable at the site of implantation.

In one embodiment, the barrier material comprises a biologic material. A “biologic material” is one that is derived from any naturally occurring source, either non-living or living. In one embodiment, the biologic material is selected from allografts, xenog rafts, autografts, and biologic matrices, e.g., extracellular matrix proteins. As used herein, “allog raft” refers to cells, tissues, or organs transplanted between members of the same species. The member of the same species may be living or nonliving. “Xenograft” as used herein refers to cells, tissues, or organs transferred between members of different species, whether living or non-living. Examples of species that commonly serve as a xenograft source include, but are not limited to, simian, porcine, bovine, ovine, equine, feline, and canine. “Autograft” as used herein refers to cells, tissues, or organs transplanted from one site to another on the same patient.

In one embodiment, the allograft, xenograft, or autograft biologic material is selected from: connective tissues, e.g., tendons, ligaments, cartilage, and fascia; musculoskeletal tissues, such as bone and muscle; cardiovascular tissues such as heart valves and blood vessels; and dermal tissue such as dermis, epidermis, and whole skin; and neural tissue. In one embodiment, the biologic material is selected from one or more of the dermis, fascia, fascia lata, pericardium, tendon, ligament, and muscle. Other tissue sources are disclosed in U.S. Pub. No. 2010/0185219, the disclosure of which is incorporated herein by reference.

In one embodiment the biologic material is selected from naturally occurring polymers. Exemplary naturally occurring polymers include fibrin, fibrinogen, elastin, graft materials, chitosan, extracellular matrix (ECM), carrageenan, chondroitin, pectin, alginate, alginic acid, albumin, dextrin, dextrans, gelatins, mannitol, n-halamine, polysaccharides, poly-1,4-glucans, starch, hydroxyethyl starch (HES), dialdehyde starch, glycogen, amylase, hydroxyethyl amylase, cellulose, cellulose derivatives such as an alkyl cellulose (e.g., ethyl cellulose) and an alkoxycellulose (e.g., hydroxypropyl cellulose), amylopectin, glucoso-glycans, fatty acids (and esters thereof), hyaluronic acid, protamine, polyaspartic acid, polyglutamic acid, D-mannuronic acid, L-guluronic acid, zein and other prolamines, alginic acid, guar gum, and phosphorylcholine, as well as co-polymers and derivatives thereof.

In one embodiment, the biologic material is a biologic matrix derived from tissue sources (e.g., soft tissue sources), including dermal, fascia, dura, pericardia, tendons, ligaments, or muscle. The biologic matrix may comprise at least one anti-infective, e.g., at least one slowed release anti-infective. Exemplary dermal matrices include, for example, acellular dermal matrices. Exemplary acellular biological material includes intact basement membrane or acellular musculoskeletal, cardiovascular, connective, dermal, and neural tissues. In one embodiment, the biologic material is a combination of cellular and acellular tissue.

In one embodiment, the biologic material is an extracellular matrix material (ECM). Exemplary ECMs include naturally-derived collagenous ECMs isolated from suitable animal or human tissue sources. Suitable extracellular matrix materials include, for instance, submucosa (including for example small intestinal submucosa, stomach submucosa, urinary bladder submucosa, or uterine submucosa, each of these isolated from juvenile or adult animals), renal capsule membrane, amnion, dura mater, pericardium, serosa, peritoneum or basement membrane materials, including liver basement membrane or epithelial basement membrane materials. These materials may be isolated and used as intact natural sheet forms, or reconstituted collagen layers including collagen derived from these materials and/or other collagenous materials may be used. Other exemplary ECMs, and methods of isolation and treatment are disclosed in U.S. Pat. No. 7,795,027, the disclosure of which is incorporated herein by reference.

In one embodiment, the biologic material is a collagen or collagen-based material can be derived from a submucosa tissue source, e.g., small intestinal submucosa, as described in U.S. Pub. No. 2010/0106257, the disclosure of which is incorporated herein by reference.

Exemplary ECMs may contain residual bioactive proteins or other ECM components derived from the tissue source of the materials. For example, they may contain Fibroblast Growth Factor 2 (basic FGF), vascular endothelial growth factor (VEGF), and Transforming Growth Factor-beta (TFG-beta). ECM base materials may contain additional bioactive components including, for example, one or more of glycosaminoglycans, glycoproteins, proteoglycans, and/or growth factors.

Exemplary extracellular matrix proteins include collagen, elastin, hyaluronic acid, and glycosaminoglycans.

In one embodiment, the biologic material comprises or is treated with at least one growth factor. Exemplary growth factors include platelet-derived growth factor (PDGF), fibroblast growth factor (FGF 1-23) and variants thereof, transforming growth factor-beta (TGF-beta) and vascular endothelium growth factor (VEGF), Activin/TGF, steroids, or any combination thereof.

In one embodiment, the biologic material comprises or is treated with at least one anti-infectant, e.g., anti-inflammatory agents, analgesic agents, local anesthetic agents, antispasmodic agents, or combinations thereof.

In one embodiment, the biologic material comprises or is treated with one or more protease inhibitors; suitable examples of protease inhibitors include Aminoethylbenzenesulfonyl fluoride HCL, Aprotinin, Protease Inhibitor E-64, Leupeptin, Hemisulfate, EDTA, Disodium (0.025-0.10 um) and trypsin-like proteases, Pepstatin A (Aspartic Proteases), Marmistat (MMP2),

In one embodiment, the barrier material comprising biologic material can comprise two or more layers. The reinforcement material can be laminated against the biologic material. The reinforcement material can comprise a biologic or non-biologic material as disclosed herein. In another embodiment, the barrier can comprise a first and third outer layer, and a second inner layer, wherein the outside layers comprises a biological material and the inside layer comprises the reinforcement material. The outer biological material layer(s) and the inner reinforcement layer may be the same size or a different size. The biological material of the first layer may be the same as the biological material of the third layer, or the biological material of the first layer may be different than the biological material of the third layer.

In one embodiment, a multiple layer biologic material may be attached to one side of the reinforcement material.

In one embodiment, the reinforcement material comprises a coated mesh as described herein and the barrier material comprises a first layer of biologic material on one side of the coated mesh and a second layer of biologic material on the other side of the mesh. In further embodiments, the layers of biologic material may be attached to the reinforcement material through means of an adhesive, coating, stitching or insert molding. In a further embodiment, the layers of biologic material may be attached to each other through openings in the reinforcement material, such as by adhesive or a lamination process involving heat or pressure or a combination of heat and pressure.

In one embodiment, the barrier material can comprise the biologic material admixed with a non-biologic material (e.g., synthetic polymers)

Polymers which may be employed to form the barrier material 160 along with the biologic material include, but are not limited to, polyethers (both substituted and unsubstituted); poly(hydroxyethyl methacrylate); polyurethanes; polyamides; polyanhydrides; polysulfones; polycaprolactones; polyglycolides; polylactides, such as, for example, polylactic acid; polyphosphazenes; poly amino acids; poly-orthoesters; polyiminocarbonates; polytrimethylene carbonates; polyhydroxymethacrylates; polyhydroxybutyrate; polyglyconate; polydioxanone; polyhydroxyvalerate; polyhydroxybutyrate/polyhydroxyvalerate copolymers; polyester urethanes; polyether urethanes, and polyurethane urea. In one embodiment, the polymer may be a copolymer formed from any combination of the above components. The polymer may also be a polymer of a “soft” component selected from the group consisting of polyethers (both substituted and unsubstituted) and poly(hydroxyethyl methacrylate) or a “hard” component selected from the group consisting of urethanes, amides, and esters. It is also contemplated that the polymer can be in the form of a hydrogel. A hydrogel, because of its hydrophilicity, invites little fibrosis, because there is little protein absorption which is involved in cell attachment and, hence, little fibrosis. Suitable materials for the barrier material 160 include but are not limited to the polymers described in U.S. Pat. No. 5,508,036 and U.S. Pat. No. 5,480,436 both of which are herein incorporated by reference in their entirety. In one embodiment, the barrier material including the biologic further comprises as a reinforcement layer or laminate, a bioabsorbable segmented copolymer comprising a first component which is a polyalkylene glycol and a second component which is a polyester formed from an alkylene glycol having from 2 to 8 carbon atoms and a dicarboxylic acid.

In one embodiment, the barrier material 160 comprises a poly lactide polymer or co-polymer and, e.g., comprises poly(L-lactide-co-D,L-lactide) 70:30 Resomer LR708 manufactured and supplied from Boehringer Ingelheim KG of Germany. Suitable materials for the barrier material include but are not limited to the materials described in U.S. Pat. No. 6,673,362 and U.S. Pat. No. 6,531,146 both of which are herein incorporated by reference in their entirety.

It is contemplated that the barrier material 160 can comprise materials such as, but not limited to SEPRAFILM® sold by Genzyme, EDICOL™ made by Devro, PELVICOL® as made by BARD and OXIPLEX® Barrier or MEDISHIELD™ made by Fziomed or similar materials.

In one embodiment, the biologic material is pressed against the coated mesh without an adhesive, possibly while applying heat and pressure to form a laminate. In another embodiment, the mesh coating can act as the adhesive to the barrier material.

In one embodiment, the barrier comprising the biologic is applied to at least one surface of a coated mesh, i.e., on one side or on both sides of the mesh.

In one alternative, the barrier material 160 may be coated with an adhesive such as, but not limited to, cellulose (such as carboxymethyl cellulose, or CMC, and hydroxypropyl methyl cellulose, or HPMC); mucoadhesives, such as, but not limited to, mucin, mucopolysaccharides, polycarbophil, tragacanth, sodium alginate, gelatin, pectin, acacia, and providone; acrylates (such as polyacrylic acid and methyl methacrylate); polyoxyethylene glycol having a molecular weight of from about 100,000 to about 4,000,000; mixtures of zinc oxide and eugenol; a fibrin-glue layer; a chitosan layer; and glucosamine. Such a coating improves initial adhesion of the barrier material 160 of the composite prosthesis 100 to tissue, such as the peritoneum.

In another embodiment, the adhesive may be admixed with the polymer in the barrier material 160 of the device, as the barrier material 160 is being formed. In such a manner, a portion of the adhesive will be exposed on the desired surface of the barrier material 160 upon formation of the barrier material.

In one embodiment, the barrier material 160 is formed of a translucent material which allows the physician to observe the location and integrity of the composite prosthesis during implantation. Holes may be formed through the barrier to facilitate passage of neutrophiclic graneulocytes, reducing the incidence of infection. The holes should have dimensions sufficient to permit neutrophilic graneulocytic transport without detrimentally affecting the adhesion resistance of the composite.

In one embodiment, the barrier material can be non-porous, or a porous material in which essentially all of the pores have a pore size no greater than 3 microns.

Other surgical adhesion resistant materials also may be used as would be apparent to those of skill in the art.

### Integration:

Referring to FIG. 2A, the coated mesh 220 and the barrier material 260 of the composite prosthesis 200 can be integrally attached or, as shown in FIG. 2B, the two layers can be separate.

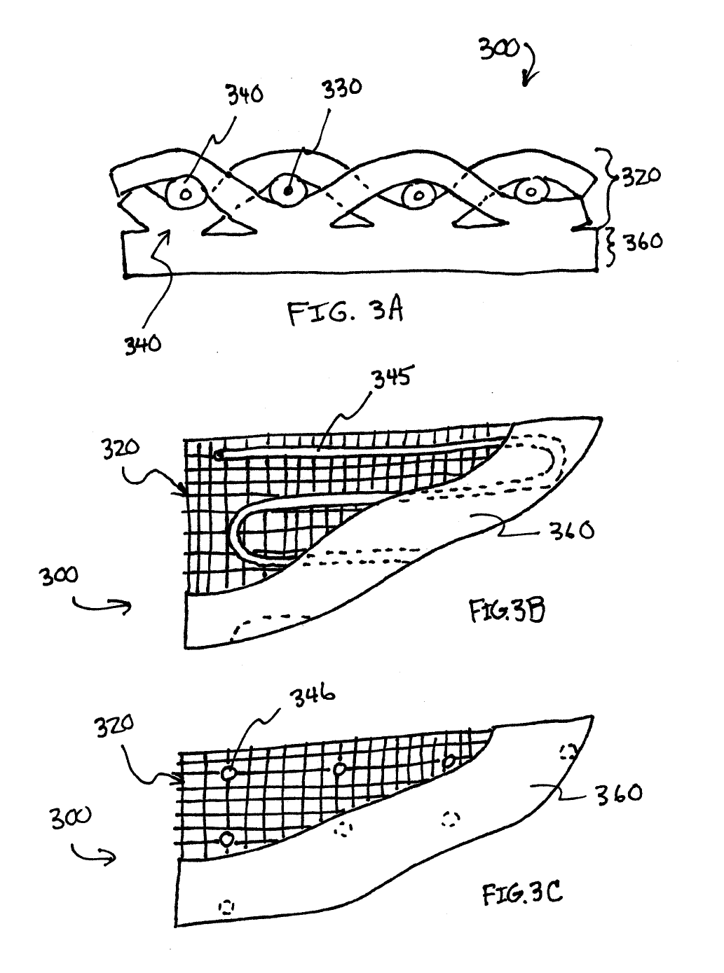

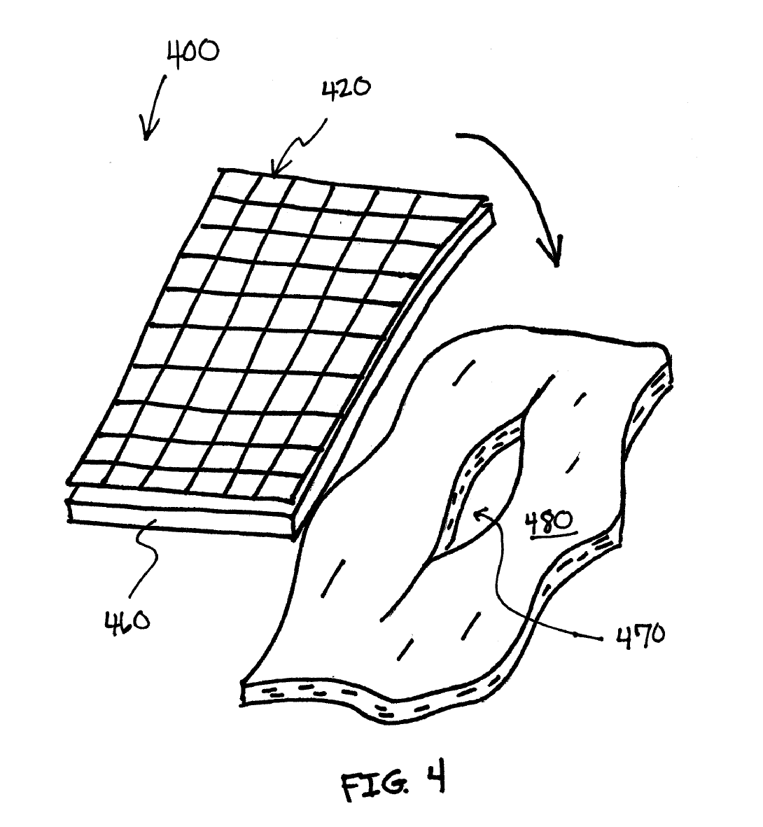

FIGS. 3A-3C show embodiments of the composite prosthesis 300 where the coated mesh 320 and the barrier material 360 are integrally attached or otherwise connected. In one embodiment shown in FIG. 3A, this connection is provided by attaching the barrier material 360 to the coated mesh 320 by using the coating 340 on the mesh fibers 330 as an adhesive. This can be accomplished by polymerizing the coating 340 on the fibers 330 or the coated mesh 320 and the barrier material 360 at the same time. An exemplary hydrogel for joining the barrier material 360 to the coated mesh 320 is the surgical adhesive described in U.S. Pat. Pub. No. 2005/0129733. These prepolymers form a hydrogel matrix when mixed with an aqueous solution which bonds to the sheet of barrier material. Other adhesives may be utilized as would be apparent to those of skill in the art, the ultimate selection depending upon the composition of the mesh and the barrier material.

By way of further example, a mesh may be coated as described in the example of U.S. Pat. Pub. No. 2006/0233852, the disclosure of which is hereby incorporated by reference, by applying the non-absorbable hydrogel-forming polyol compositions, as disclosed herein, in solution to a mesh (with or without openings pre-formed in the mesh as contemplated in U.S. Pat. Pub. No. 2006/0233852) and, prior to curing of the coating, applying the barrier material to the coated uncured mesh. As the coating cures, it will also adhere the barrier material to the coated mesh. If the barrier layer is to be applied to both sides of the coated mesh, a first barrier layer may be applied to one side of the uncured coated mesh before the second barrier layer is applied to the other side of the coated mesh. The second barrier layer may be applied while the coated mesh is substantially uncured, with the coated mesh then cured to attach both layers to the coated mesh. Alternatively, the first barrier layer may be applied while the coated mesh is uncured, with the second barrier layer applied after the coated mesh has cured with the first layer adhered to the coated mesh, such as by use of an adhesive, stitching, etc.

Alternatively as shown in FIGS. 3B and 3C, additional adhesive may be applied in a grid-like pattern of dots or beads. In a representative arrangement shown in FIG. 3C, spaced dots 346 form an effective joint between the coated mesh 320 and the barrier material 360. A serpentine pattern 345 as shown in FIG. 3B or staggered configurations are also possible. Various other shapes, sizes and patterns of adhesives or other attachment means such as, but not limited to sewing, may be used as would be apparent to those of skill in the art.

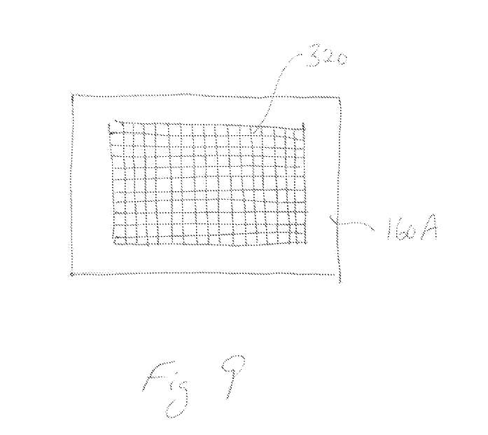

In a further embodiment coated mesh 320 may be partially covered by one or more barrier layers. By way of example, referring to FIG. 8, coated mesh 320 may be assembled with a first barrier layer 160A and a second barrier layer 160B, such that only the peripheral edges of the coated mesh are covered by the barrier layer. In such a construct the coated mesh is uncovered in part and covered in part. See, for example, FIG. 9, illustrating a top view of a coated mesh 320 with a central portion of the mesh exposed and uncovered and the peripheral edges of the coated mesh covered by barrier material 160A. The barrier layer covering only a portion of the coated mesh may be applied to one or both sides of the coated mesh. Alternatively, a layer of barrier material only partially covering the coated mesh may be applied to one side of the coated mesh, with a layer of barrier material applied to the other side of the coated mesh covering all or substantially all of the other side of the coated mesh.

Embodiments of the present invention therefore provide a composite prosthetic, amongst which are certain of the following advantages. The composite prosthesis combines the strength of a mesh material and the low adhesion incidence of a physical barrier. If a coated mesh with openings as contemplated in U.S. Pat. Pub. No. 2006/0233852, the disclosure of which is hereby incorporated by reference, is used, after a bioabsorbable barrier has been absorbed or resorbed, the composite may be anchored in place by tissue through-growth through the coated mesh openings after the barrier layer is absorbed by the body. The specific pattern of attachment (adhesive, molding, stitching, etc.) of the mesh and barrier material can provide a dimensional strength to the prosthesis.

The composite can be used for repair of ventral hernias (incisional and umbilical) and chest wall defects where it is more common for the prosthetic mesh to be exposed to the abdominal viscera due to insufficient or unavailable autogenous tissue. The non-inflammatory coating prevents the mesh fabric from causing inflammation of the abdominal viscera, reducing the incidence of intestinal adhesion and fistulization. It also is contemplated that the composite prosthesis would be indicated for use in laparoscopic procedures, e.g., intraperitoneal applications where the peritoneum would not be available to provide a natural barrier between the implant and the intestine.

It is contemplated that embodiments of the prosthesis can be sized and shaped to be used with specific surgical procedures that require uniquely sized and shaped embodiments of the present invention.

Other embodiments of the composite prosthesis comprise the herein described embodiments of a coated mesh and absorbable barrier material having an additional non-absorbable layer of barrier material. This non-absorbable layer of barrier material is placed on the surface of the coated mesh opposite of the surface against which the barrier material is placed. Examples of suitable materials to create the nonabsorbable barrier material include, but are not limited to those non-absorbable prepolymers described in U.S. Pat. Pub. No. 2006/0233852, the disclosure of which is hereby incorporated by reference.

The device may also contain pharmaceutical agents (e.g., proteins, nucleic acids, antibiotics, etc.) which are placed in the device with an acceptable pharmaceutical carrier. Such agents may diffuse out of the device and into the body after implantation, and/or may be released internally upon degradation of the device, thereby providing a desired therapeutic effect.

### Method of Operation:

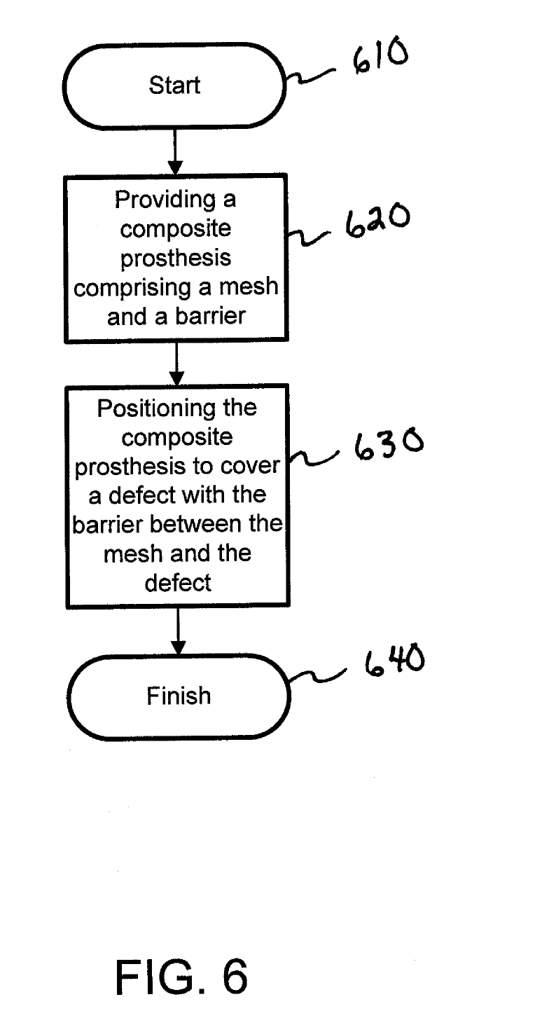

FIG. 6 is a process diagram illustrating one embodiment of a method of using the composite prosthesis. The method starts with step 610 and is followed by step 620 of providing a composite prosthesis comprising a coated mesh and a barrier material. Step 630 comprises positioning the composite prosthesis to cover the defect. The method is concluded with step 640.

This embodiment is further illustrated by, but not limited to, the embodiment in FIG. 4. Referring to FIG. 4, the composite prosthesis 400 can be used to repair a defect 470 in a wall 480 of a patient's body cavity that exposes a visceral surface. To repair the defect 470, a medical professional inserts the composite prosthesis 4400 through opening 470 and into the body cavity. The composite prosthesis 400 is positioned such that coated mesh surface 420 faces the visceral surface and the barrier material 460 faces the tissue wall 480 and covers the defect 470. Once the composite prosthesis 400 is positioned, the medical professional attaches the composite prosthesis 400 to the tissue surrounding the defect 470 with sutures, staples, tacks, glue or other attaching means.

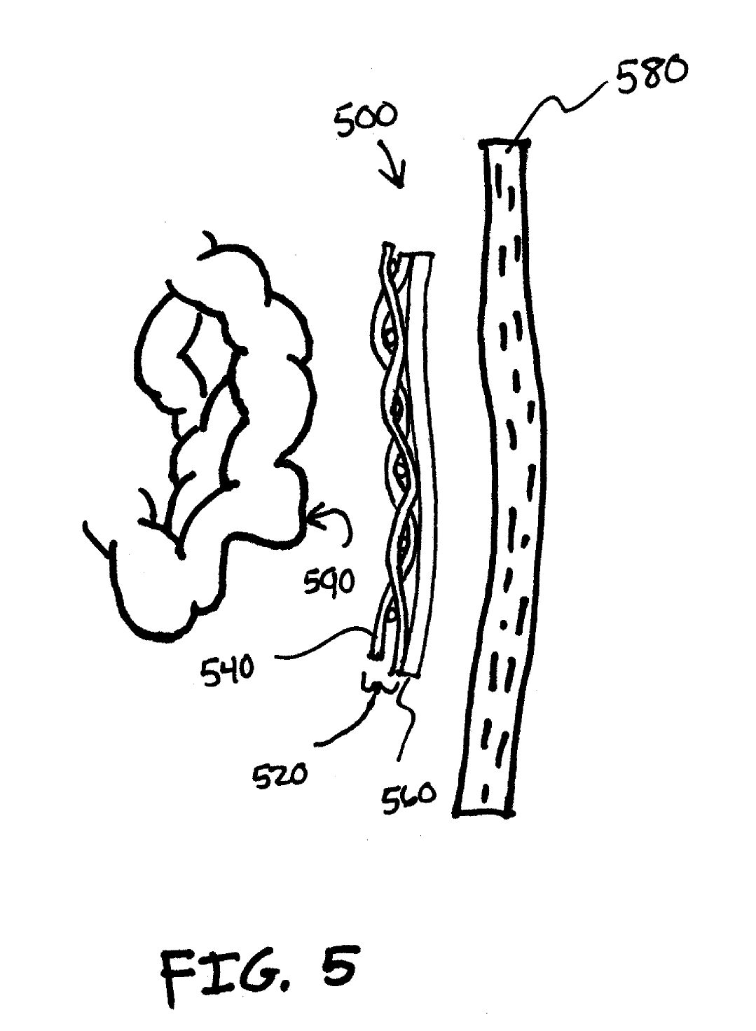

As used in the repair of ventral hernias and in chest wall reconstruction, as illustrated in FIG. 5, the barrier material 560 isolates the abdominal viscera 590 from the healing process of the defect and the coated mesh 520 reduces the inflammatory response characteristic of the mesh fibers. Together, these features prevent intestinal adhesion and fistulization which may result from an inflammatory reaction of the visceral surface 590 and the mesh throughout the healing process.

Referring to FIG. 5, as part of the normal healing process, the barrier material 560 is absorbed by the body, leaving the coated mesh 520 between the tissue 580 and the visceral surface 590. During this healing, the barrier material 560 is placed against the defect, and because it is a hydrogel, it minimizes the stimulation of the defect tissue. By the time the barrier material 560 has been absorbed, the defect in the tissue 580 has healed to an extent (e.g., a new peritoneal surface has formed over the defect) that the likelihood of adhesions forming between viscera 590 and coated mesh 520 is minimal. The barrier material 560 may be absorbed by the body over a period of at least about 14 days from an initial implantation of the prosthesis. In addition to the adhesion resistance provided by the barrier material 560, the coating 540 covering the mesh and mesh fibers provides a second defense against adhesion prevention by further minimizing the stimulation of the defect tissue 580.

After the barrier material 560 is absorbed by a patient's body, the coated mesh 520 of the composite prosthesis 500 becomes incorporated into the tissue wall 580 by in-growth (if the coated mesh is constructed to permit such in-growth), or by through-growth of tissue 580 through the openings or windows formed in the coated mesh 520 for that purpose as described in U.S. Pat. Pub. No. 2006/0233852, the disclosure of which is hereby incorporated by reference, in which tissue grows through the openings and around or over the coated mesh rather than into the interstices of the mesh, the mesh coating or the mesh fibers. While implantation with the barrier material 560 facing the defect to be repaired is described herein, it is also contemplated that the barrier material 560 could be implemented facing the viscera 590, with the coated mesh 520 facing the defect. As will be appreciated, any of the embodiments described herein may be used for tissue repair, with the barrier material disposed on one or both surfaces of the coated mesh covering all, substantially all or part of one or both sides of the coated mesh.

Examples of uses of the devices include, but are not limited to, barriers and prostheses between the internal female reproductive organs (e.g., uterus, fallopian tubes, ovaries); barriers and prostheses between the internal female reproductive organs and the peritoneum; barriers and prostheses for used during laparoscopy; barriers and prostheses between periodontal tissue; barriers and prostheses between cartilages or between cartilage and bone; barriers and prostheses between digestive organs; spinal barriers and prostheses; barriers and prostheses between digestive organs and peritoneum; barriers and prostheses between the epicardium and surrounding structures such as the pericardium, mediastinal fat, pleura, and sternum; barriers between tendons and tendon sheaths, such as those in the wrist and ankle; bone fracture wraps; barriers between muscle tissue and bone; barriers and prostheses between the esophagus and mediasternum; barriers and prostheses between the gall bladder or pancreas and the peritoneum; and barriers and prostheses for scrotal surgery.

### Making the Composite Apparatus:

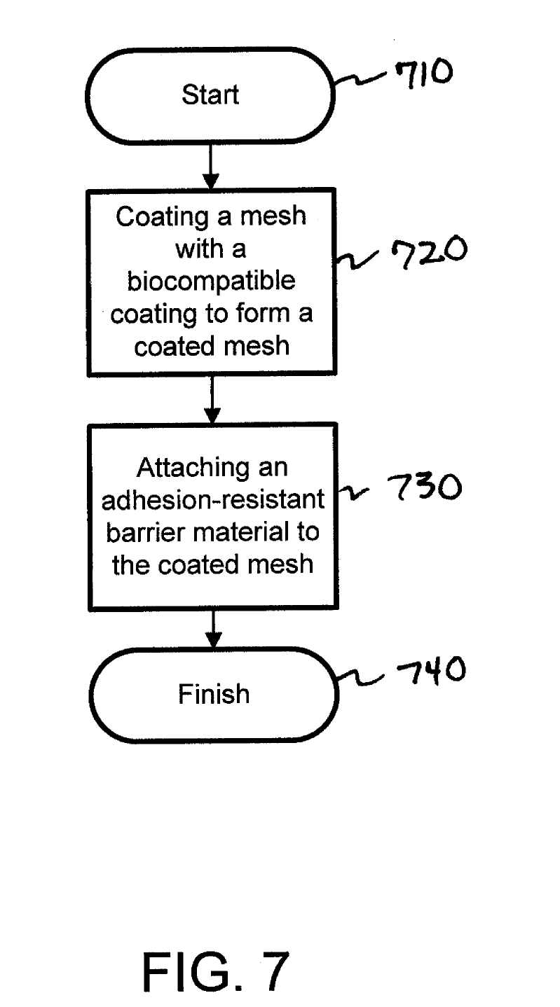

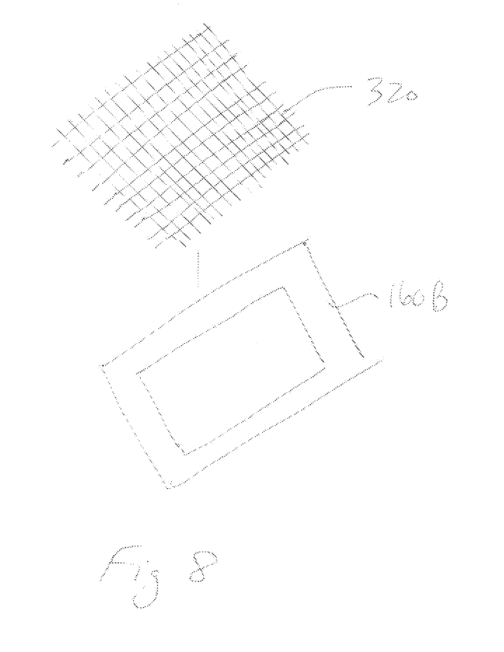

FIG. 7 illustrates one embodiment of a method of making the composite prosthesis. This embodiment starts with step 710 and is followed by step 720 comprising a step of coating a mesh with a biocompatible coating to form a coated mesh. Next, step 730 comprises attaching the adhesion-resistant barrier material to the coated mesh. The method is complete with step 740. The composition of the mesh, biocompatible coating and barrier material compositions include, but are not limited to those described and referenced herein.

The method described above illustrates one method of creating several embodiments of the composite prosthesis to include, but not limited to those shown in FIGS. 1, 2A, 3B, 3C, 8 and 9.

Other embodiments of the method described are contemplated, including but not limited to methods of making the embodiment illustrated in FIG. 2B, where the coated mesh and the barrier material are made separately.

In other embodiments, including but not limited to the embodiment illustrated in FIG. 3A, the step of coating the mesh and the step of attachment of the coated mesh to the barrier material can occur nearly simultaneously or within a sufficient time period that the barrier material can be applied to the coated mesh before the coating has cured and thereby attaching the barrier to the coated mesh. In a multi-step coating process, the barrier layer may be applied before the last coating layer has cured.

In one embodiment, the biologic material may be in a dry or wet state during application. If applied in a dry state, the final composite mesh may be hydrated and maintained in a hydrated state in the package. In another embodiment, the composite mesh can be shipped in a dry state and rehydrated prior to use in surgery.

With respect to the above description then, it is to be realized that the optimum chemical and mechanical relationships include variations in size, materials, shape, form, function and manner of operation, assembly and use, are deemed readily apparent and obvious to one skilled in the art, and all equivalent relationships to those illustrated in the drawings and described in the specification are intended to be encompassed by the present disclosure.

Therefore, the foregoing is considered as illustrative only of the principles of the invention. Further, since numerous modifications and changes will readily occur to those skilled in the art, it is not desired to limit the invention to the exact embodiment and operation shown and described, and accordingly, all suitable modifications and equivalents may be resorted to, falling within the scope of the invention. Although this invention has been described in the above forms with a certain degree of particularity, it is understood that the present disclosure has been made only by way of example and numerous changes in the details of construction and combination and arrangement of parts may be resorted to without departing from the spirit and scope of the invention.

Other embodiments of this invention are within the scope of the following claims.

1. A composite prosthesis comprising:
a coated mesh having at least one opening through a first surface and a second surface of the coated mesh;
the coated mesh comprising a mesh and a biocompatible coating substantially surrounding each filament of the mesh, wherein the biocompatible coating is formed by coating the mesh with a polyol prepolymer and curing the prepolymer, the prepolymer comprising a polyalkylene oxide polyol end capped with isocyanate, the polyalkylene oxide polyol having from about 70% to about 95% ethylene oxide groups and the remainder propylene oxide; and
a barrier material comprising a biocompatible membrane constructed and arranged to cover at least one surface of the coated mesh, wherein the barrier material comprises a biologic material.
